(19) United States
(12) Patent Application Publication
(10) Pub. No.: US 2006/0135790 A1
(43) Pub. Date: Jun. 22, 2006

(54) Process for the preparation of an alpha-amino carbonyl compound

(76) Inventors: David John Hyett, Sittard (GB); Daniel Mink, Eupen (DE); Quirinus Bernardus Broxterman, Munstergeleen (NL); Bernardus Kaptein, Sittard (NL); Hubertus Josephus Marie Zeegers, Baarlo (NL)

Correspondence Address: NIXON & VANDERHYE, PC, 901 NORTH GLEBE ROAD, 11TH FLOOR, ARLINGTON, VA, 22203, US

(21) Appl. No.: 10/546,212

(22) PCT Filed: Mar. 4, 2004
(86) PCT No.: PCT/NL04/00162

§ 371 Date: Feb. 24, 2006

(30) Foreign Application Priority Data
Mar. 6, 2003 (EP) 0310552.3

(51) Int. Cl. C07D 207/02

(52) U.S. Cl. 548/530; 558/410

(57) ABSTRACT

The invention relates to a process for the preparation of an α-amino carbonyl compound by reacting an imine starting material with a suitable electrophile in the presence of a base. This process has the advantage that the imine starting materials can be prepared from glyoxylic acid esters or glyoxylic acid ester derivatives and α-hydrogen containing primary amines, which are usually cheap and readily available. These imine starting materials can usually be prepared with a high yield and/or almost without the formation of any side products.

The invention relates to a process for the preparation of an α-amino-carbonyl compound of formula 1, 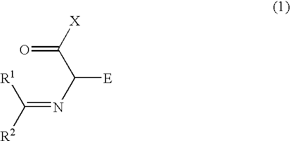
wherein R¹ and R² each independently stand for optionally substituted (cyclo)alkyl, optionally substituted (cyclo)alkenyl, optionally substituted (hetero)aryl, CN or C(O)R⁶, —wherein R⁶ stands for OR¹²—, —wherein R¹² stands for an optionally substituted (cyclo)alkyl, an optionally substituted aryl- or wherein R⁶ stands for NR¹³R¹⁴, —wherein R¹³ and R¹⁴ are each independently chosen from the group of H, optionally substituted (cyclo)alkyl and optionally substituted (hetero)aryl and wherein R¹³ and R¹⁴ may form a ring together with the N-atom to which they are connected- and wherein R¹ and/or R² may be part of a ring system formed by a connection between R¹ and R², between R¹ and E, between R² and E, between R¹ and X or between R² and X, wherein X and E are as defined below,
wherein E stands for H, an optionally substituted (cyclo)alkyl, a halogen, a tri-substituted silyl group, an optionally substituted (cyclo)alkenyl, an optionally substituted (hetero)aryl or wherein E stands for C(O)R⁴⁰, —wherein R⁴⁰ stands for H, an optionally substituted (cyclo)alkyl, an optionally substituted (hetero)aryl or for OR⁴¹, —wherein R⁴¹ stands for an optionally substituted (cyclo)alkyl or an optionally substituted (hetero)aryl or wherein R⁴⁰stands for NHR⁴²—, —wherein R⁴² stands for H, an optionally substituted (cyclo)alkyl or for an optionally substituted aryl-,
and wherein X stands for OR⁵, —wherein R⁵ stands for an optionally substituted (cyclo)alkyl, an optionally substituted aryl- or wherein X stands for NR³R⁴, —wherein R³ and R⁴ each independently stand for H, an optionally substituted (cyclo)alkyl or an optionally substituted (hetero)aryl and wherein R³ and R⁴ may form a ring together with the N-atom to which they are bound-,
and wherein X together with E may form part of a lactone or lactam ring system together with the C-atoms to which they are bound.

A process for the preparation of a compound of formula 1, wherein R¹ and R² stand for phenyl, has been disclosed by O'Donnell and is reviewed in O'Donnell et al, Aldrichimica Acta (2001) vol. 34, pp 3-15. In this process of O'Donnell, the α-amino carbonyl compound is prepared by deprotonation of a starting material and subsequent reaction with an electrophile. The starting materials in this O'Donnell process are imines derived from benzophenone and glycine esters or from benzophenone and glycine amides.

However, a drawback of this process is that the preparation of these imine starting materials is commercially less attractive. One possible synthetic route involves the direct reaction of a glycine ester with benzophenone. However, due to the low reactivity of benzophenone this method requires the use of a strong Lewis acid catalyst (e.g. BF₃.Et₂O). In addition to the toxicity of such reagents, this methodology also results in low product yields (due to the formation of side-products) and renders the purification of the product difficult. Another possible route to prepare these imine starting materials is via a transimination reaction. In this case the hydrochloride salt of a glycine ester is reacted with benzophenone imine. However, benzophenone imine must itself be prepared by the addition of an organometallic reagent to benzonitrile, which makes this a commercially less attractive alternative. Processes involving the use of such imine starting materials are therefore not very suitable for large scale commercial production.

It is the object of the invention to provide a process for the preparation of an α-amino-carbonyl compound of formula 1 from a starting material, which starting material may be prepared by a commercially attractive route.

This object is achieved by a process wherein an imine of formula 2, 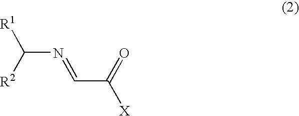
wherein R¹, R² and X are as defined above, is reacted with a suitable electrophile in the presence of a base to form the corresponding a-amino carbonyl compound of formula 1.

If R¹ and/or R² stand(s) for an optionally substituted (hetero)aryl, preferably the (hetero)aryl including the substituent(s) contains 1-20 C-atoms, for example, an optionally substituted phenyl group or an optionally substituted naphthyl group, more preferably the (hetero)aryl including the substituent(s) contains 3-15 C-atoms, more preferably 3-10 C-atoms, for example a phenyl group. Preferably the heteroaryl is an aromatic system containing one or more heteroatoms chosen from the group of N, O and S. If R¹ and/or R² stand(s) for an optionally substituted (cyclo)alkyl, preferably the (cyclo)alkyl including the substituent(s) contains 1-10 C-atoms, more preferably 1-8 C-atoms, for example a methyl group. If R¹ and/or R² stand(s) for an optionally substituted (cyclo)alkenyl, preferably, the (cyclo)alkenyl including the substituents contains 2-10 C-atoms, more preferably 2-8 C-atoms, for example a vinyl group. R¹ and R² may form a ring together with the C-atom to which they are bound of preferably 3-8 atoms, more preferably of 5-6 atoms, for example, R¹ and R² together with the C-atom to which they are bound may form a cyclohexyl ring, or a 9-fluorenyl group.

If R¹² and/or R¹³ and/or R¹⁴ stand(s) for an optionally substituted (cyclo)alkyl, preferably the (cyclo)alkyl including the substituent(s) contains 1-10 C-atoms.

If R¹² stands for an optionally substituted aryl, preferably the aryl including the substituent(s) contains 1-20 C-atoms, more preferably 3-15 C-atoms, most preferably 3-10 C-atoms.

Preferably R¹ and R² each independently stand for optionally substituted (cyclo)alkyl, optionally substituted (cyclo)alkenyl, optionally substituted (hetero)aryl, wherein R¹ and/or R² may be part of a ring system formed by a connection between R¹ and R², between R¹ and E, between R² and E, between R¹ and X or between R² and X, wherein X and E are as defined above.

More preferably, R¹ and R² each independently stand for an optionally substituted (cyclo)alkyl or an optionally substituted (hetero)aryl and wherein R¹ and R² may form part of a ring system formed by a connection between R¹ and R².

Preferably X stands for OR⁵, wherein R⁵ stands for an optionally substituted (cyclo)alkyl of preferably 1-10 C-atoms, more preferably 1-8 C-atoms (substituents included) or X stands for NR³R⁴, wherein R³ and R⁴ each independently stand for H, an optionally substituted (cyclo)alkyl of preferably 1-10 C-atoms, more preferably 1-8 C-atoms (substituents included) or an optionally substituted aryl of preferably 5-6 C-atoms, wherein R³ and R⁴ may form a ring of preferably 3-8 atoms, more preferably of 5-6 atoms, together with the N-atom to which they are bound, and wherein X together with E may form part of a lactone or lactam ring system of preferably 5-6 atoms, together with the C-atoms to which they are bound.

Preferably E stands for H or an optionally substituted (cyclo)alkyl of preferably 1 to 30 C-atoms, and E together with X may form part of a lactone or lactam ring system of preferably 5-6 atoms, together with the C-atoms to which they are bound.

If R⁴⁰ and/or R⁴² stand(s) for an optionally substituted (cyclo)alkyl, preferably the (cyclo)alkyl including the substituent(s) contains 1-20 C-atoms. If R⁴⁰ and/or R⁴² stand(s) for an optionally substituted (hetero)aryl, preferably the (hetero)aryl including the substituents contains 1-20 C-atoms.

If R⁴¹ stands for an optionally substituted (cyclo)alkyl, preferably the (cyclo)alkyl including the substituent(s) contains 1-10 C-atoms. If R⁴¹ stands for an optionally substituted (hetero)aryl, preferably the (hetero)aryl including the substituent(s) contains 3-10 C-atoms.

Examples of optional substituents for E and R⁴⁰ include: a (hetero)aryl group, an alkenyl group, an alkynyl group, an alkoxy group, an aryloxy group, a carbonate group, a cyano group, a (masked) ketone group, preferably a (cyclic) ketal, a (masked) aldehyde group, preferably a (cyclic) acetal, a carboxylic acid ester group, a carboxylic acid amide group, an amino group, a (di)alkylamino group, a (di)aryl amino group, an (aryl)(alkyl)amino group, a halogen, a trisubstituted silyl group, a silyloxy group, a phosphonate group, a sulphonate group, a thioether group, a sulfoxide group, a sulfone group, a hydroxy group, an acyloxy group, an acylamido group, a nitro group, a carbamoyl group, a guanidyl group or a thiol group.

The tri-substituted silyl group may be a silyl group substituted with an alkyl (of preferably 1 to 6 C-atoms) and/or an aryl (of preferably 3 to 6 C-atoms), for example the tri-substituted silyl group is tri-methyl silyl.

Examples of optional substituents for R¹, R², R³, R⁴, R¹³, R¹⁴ and R⁴² include a (hetero)aryl group, an alkenyl group, an alkynyl group, an alkoxy group an aryloxy group, a cyano group, a (masked)ketone group, preferably a (cyclic) ketal, a (masked) aldehyde group, preferably a (cyclic) acetal, a carboxylic acid ester group, a carboxylic acid amide group, an amino group, a (di)alkylamino group, a (di)arylamino group an (aryl)(alkyl)amino group, a halogen, a thioether group, a hydroxy group, an acyloxy group, an acylamido group, a carbamoyl group, a guanidyl group, a nitro group or a thiol group.

Examples of optional substituents for R⁵, R¹² and R⁴¹ include: a (hetero)aryl group, an alkenyl group, an alkynyl group, an alkoxy group, an aryloxy group, a cyano group, a ketone group, a (masked) aldehyde group, preferably a (cyclic) acetal, a carboxylic acid ester group, a carboxylic acid amide group, a dialkylamino group, a diarylamino group, an (aryl)(alkyl)amino group, a halogen, a thioether group, a hydroxy group, an acyloxy group, an acylamido group, a carbamoyl group, a guanidyl group, a nitro group or a thiol group.

Electrophiles suitable for the introduction of E into a compound of formula 2 include, for example, proton sources, for example H₂O or methanol; non-activated alkyl halides, in particular non-activated alkyl iodides, for example n-butyl iodide; propargylic halides, for example propargyl bromide; allylic halides, for example allyl bromide; 1-arylalkyl halides, for example benzyl bromide; Michael acceptors (which can be defined as alkenes activated towards nucleophilic attack by the presence of an electron withdrawing group), for example acrylonitrile, methyl acrylate and chalcone; carboxylic acid chlorides, for example acetylchloride; carboxylic acid anhydrides, for example acetic anhydride; activated carboxylic acid esters, for example pentafluorophenol esters, N-hydroxysuccinimide esters or N-hydroxybenzotriazol esters; epoxides and aziridines; alcohol groups that have been activated towards substitution, e.g. tosylates, mesylates, triflates or nosylates; electrophilic sources of halogens, for example N-chloro- or N-bromo succinimide; silylating reagents, for example trimethylsilylchloride; (masked) aldehydes; ketones, aldimines; ketimines; isocyanates; chloroform ate esters.

The choice of temperature for the conversion of a compound with formula 2 into a compound with formula 1 is in principle not critical, for example, temperatures ranging from −80° C. to 80° C. may be employed. Preferably, temperatures for said conversion are from −5 to 35° C.

The invention includes different embodiments with different conditions, which can be employed for the preparation of an α-amino carbonyl compound of formula 1. For example, in one aspect of the invention a compound of formula 1 can be prepared from a compound of formula 2 by reacting a compound of formula 2 with a suitable electrophile in the presence of a base in an anhydrous organic solvent.

Examples of bases that can be used in the preparation of a compound of formula 1 from a compound of formula 2 in an anhydrous organic solvent include: alkali metal alkoxides, for example potassium tert-butoxide; alkalimetal hydrides, for example sodium hydride; organo lithiums, for example n-butyl lithium; alkali metal amides, for example lithium diisopropylamide or lithium hexamethyidisilazide, potassium hexamethyidisilazide or sodium hexamethyldisilazide; guanidines, for example tetramethylguanidine; phosphazenes, for example Schwesinger Phosphazene Base P₁-t-butyl-tris(tetramethylene) (BTPP). Preferably a base, which corresponding conjugated acid has a pKₐ>10, more preferably a pKₐ>13, most preferably a pKₐ>15, is used in the preparation of a compound of formula 1 from a compound of formula 2 in an anhydrous organic solvent.

The specific choice of anhydrous organic solvent is, in principle, not critical. Examples of solvents which may be used in the conversion of a compounds of formula 2 into a compound of formula 1 include: dialkyl ethers, for example methyl tert-butyl ether or tetrahydrofuran; halogenated solvents, for example dichloromethane; hydrocarbons, for example toluene; alcohols, for example tert-butanol;

In another aspect of the invention, the preparation of a compound of formula 1 from a compound of formula 2 can be achieved by reacting a compound of formula 2 with a suitable electrophile in the presence of a base and a phase transfer catalyst in a two-phase system. Most commonly used two-phase systems are liquid-liquid systems or solid-liquid systems. Examples of liquid-liquid systems include: organic solvent-(concentrated) NaOH solution, wherein the organic solvent may for example be a hydrocarbon, for example toluene; a halogenated solvent, for example CH₂Cl₂ or chlorobenzene; or a dialkylether, for example diethylether. Examples of solid-liquid systems include K₂CO₃-acetonitrile; CsOH.H₂O in a halogenated solvent; Cs₂CO₃ in a halogenated solvent, for example CH₂Cl₂ or chlorobenzene; Cs₂CO₃ in a dialkylether, for example diethylether; Cs₂CO₃ in a hydrocarbon, for example toluene.

Suitable phase transfer catalysts include for instance quaternary ammonium or phosphonium salts, crown ethers or cryptands, as described in EV Demhlow & SS Demhlow; “Phase Transfer Catalysis”, 3rd edition, Wiley VCH Verlag, 1993.

In a special aspect of the invention, the invention relates to a process for the preparation of an enantiomerically enriched compound of formula 1 from a compound of formula 2 by reacting a compound of formula 2 with a suitable electrophile in the presence of a base and a chiral and enantiomerically enriched phase transfer catalyst in a two-phase system. Preferably the enantiomerically enriched phase transfer catalyst has an enantiomeric excess (e.e.) >90%, more preferably >95%, most preferably >98%. Many enantiomerically enriched compounds are important building blocks in the synthesis of drugs.

Chiral and enantiomerically enriched phase transfer catalysts are known in the art and include, for example, derivatives of N-alkylated cinchona alkaloids (for instance described in WO95/06029). Suitable examples of chiral and enantiomerically enriched phase transfer catalysts for these types of reactions are for instance described in the following references: M. O'Donnell, Aldrichimica Acta (2001) 34, 3-15; B. Lygo, Tetrahedron Lett. (1997) 38, 8597-8600; E. J. Corey, J. Am. Chem. Soc. (1997) 119, 1241412415; M. Shibasaki, Tetrahedron Lett. (2002) 43, 9539-9543.

In a special aspect of the invention, a diasteromerically enriched compound of formula 1 may be prepared by reacting a compound of formula 2 having one or more chiral groups with a suitable electrophile in the presence of a base. The chiral group(s) may be removed after effecting its diastereomeric induction. In case that the compound of formula 2 having the chiral group(s) is enantiomerically enriched, the resulting compound of formula 1 may be obtained both diastereomerically enriched and enantiomerically enriched. Preferably the enantiomerically enriched compound of formula 2 has >90% e.e., more preferably >95% e.e., most preferably >98% e.e. If the compound of formula 2 has more than one chiral group, it is preferred that all chiral groups are enantiomerically enriched. Especially attractive is the case wherein the compound of formula 2 is enantiomerically enriched and wherein X stands for a chiral group, which chiral group is derived from a chiral alcohol R⁵OH or from a chiral amine HNR³R⁴, wherein R³, R⁴ and R⁵ are as defined above.

The use of an enantiomerically enriched compound with a chiral group, in order to obtain a diastereomerically and enantiomerically enriched compound is for instance described by C. Nájera, Angew. Chem. (1997) 36, 995-997; A. López, Tetrahedron Asymm. (1998) 9, 1967-1970; C. Nájera, Tetrahedron Asymm. (1998) 9, 3935-3938; and Y. S. Park, Bull. Korean Chem. Soc. (2001) 22, 958-962.

A compound of formula 2, wherein X stands for OR⁵, wherein R⁵ is as defined above, can, for instance, easily be prepared by reacting a glyoxylic acid ester (derivative) of formula 3, 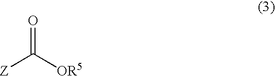
wherein Z is CHO or a masked aldehyde group, with an amine of formula 4, 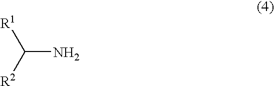
wherein R¹ and R² are as defined above. This process for the preparation of a compound of formula 2, wherein X stands for OR⁵, wherein R⁵ is as defined above, is a cheap and commercially attractive process, due to a combination of beneficial effects, for instance, glyoxylic acid esters and glyoxylic acid ester derivatives of formula 3 are cheap and readily available and/or the process proceeds with a high yield and/or the process proceeds almost without the formation of any side products.

A compound of formula 2, wherein X stands for NR³R⁴, wherein R³ and R⁴ are as defined above, can, for instance, easily be prepared by further reacting the imine of a glyoxylic acid compound of formula 2, wherein X stands for OR⁵, wherein R⁵ is as defined above with an amine of formula 5, 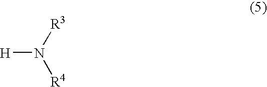
wherein R³ and R⁴ are as defined above.

The ease with which this amidation reaction occurs represents a particularly surprising aspect of the invention since the reaction of amines with carboxylic acid esters is normally a slow process and often requires, for example, the use of a high concentration of amines (the equivalent of high pressure in the case of ammonia), and/or high temperatures and/or activating agents and/or catalysts. The combination of this amidation reaction and the subsequent reaction with an electrophile is especially advantageous in the synthesis of peptides (in formula 1, X stands for NR³R⁴, wherein NR³R⁴ stands for an amino acid ester, an amino acid amide, an amino nitrile or for an N-terminus of a peptide. The peptide might be bound, for example, to a solid phase resin). This is especially advantageous, since the reaction of the amino group of amino acid derivatives or of peptides with carboxylic acid esters is normally particularly slow.

In a special aspect of the invention, the compound of formula 4 and the compound of formula 5 are the same. In this case, the compound of formula 2, wherein X stands for NR³R⁴, —wherein R³ stands for H and R⁴ stands for HCR¹R²₁, —wherein R¹ and R² are as defined above-, can be prepared directly by reacting the compound of formula 3 with the compound of formula 4.

In the preparation of a compound of formula 2, a masked aldehyde group is defined as a group which performs a similar function as an aldehyde group in this preparation or which can form an aldehyde group in situ. Examples of masked aldehyde groups include: hydrates, hemiacetals, (cyclic)acetals and bisulfite adducts.

Examples of solvents for the preparation of a compound of formula 2 wherein X stands for OR⁵ and R⁵ is as defined above include hydrocarbon solvents, for example toluene; halogenated solvents, for example dichloromethane; dialkyl ethers, for example methyl tert-butyl ether (MTBE), tetrahydrofuran, 1,2-dimethoxyethane; carboxylic acid esters, for example n-butyl acetate, i-propylacetate, ethylacetate; ketones, for example butanone or methyl isobutyl ketone (MIBK); alcohols, for example t-butanol. Preferably, temperatures for the preparation of a compound with formula 2 wherein X stands for OR5 and R⁵ is as defined above are from 0-150° C., more preferably from 0-120° C., most preferably from 0-60° C.

An α-amino carbonyl compound of formula 1, wherein X stands for OR⁵, wherein R⁵ is as defined above, may be further reacted, for example, with an amine of formula 5, 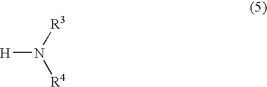
wherein R³ and R⁴ are as defined above to form the corresponding α-amino carbonyl compound of formula 1 wherein X stands for NR³R⁴, wherein R³ and R⁴ are as defined above. This is a very favorable reaction as usually hardly any side product formation occurs. Preferably in this conversion, R⁵ stands for methyl as this gives a facile conversion.

The conversion of a compound of formula 2, wherein X stands for OR⁵, wherein R⁵ is as defined above, or of a compound of formula 1, wherein X stands for OR⁵, wherein R⁵ is as defined above, into a compound of formula 2, or, respectively, into a compound of formula 1, wherein X stands for NR³R⁴, wherein R³ and R⁴ are as defined above can either be carried out in the neat amine of formula 5 or in a suitable solvent. Suitable solvents include alcohols, for example methanol, ethanol or isopropanol; carboxylic acid esters, for example ethyl acetate, isopropyl acetate and n-butyl acetate; ketones, for example butanone or MIBK; ethers, for example methyl tert-butyl ether; halogenated solvents; and hydrocarbons, for example toluene. Preferably, the temperature for these conversions is from 0-120° C.

An α-amino carbonyl compound of formula 1 may be further converted, for example, to form the corresponding compound of formula 6 or a salt thereof 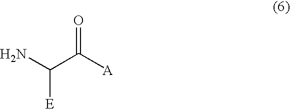
wherein A stands for OH or X and wherein X and E are as defined above, in a manner known per se.

There are several ways known by the person skilled in the art to achieve the conversion of an α-amino carbonyl compound of formula 1 into the corresponding compound of formula 6 or a salt thereof. Examples include reactions carried out under acidic, neutral and basic conditions. Conversion under acidic conditions can, for example, be performed with an aqueous mineral acid, for example 0.2-1 M HCl solution at ambient temperature, solution of concentrated aqueous HCl in acetone or with an organic acid in an aqueous solvent, for example 15% citric acid in water. Conversion under basic conditions can, for example, be performed by transimination, for example using a solution of hydroxylamine HCl. Examples of each procedure can be found in M. O'Donnell, Aldrichimica Acta (2001) 34, 3-15 and references therein. If R¹ and/or R² stand(s) for aryl, conversion under neutral conditions can, for example, be performed by hydrogenolysis, for example using a Pd/C catalyst in the presence of either hydrogen gas or ammonium formate. In the latter case, the conversion of a compound of formula 1 to the corresponding amino acid derivative of formula 6, may, for example, in the case that R¹ and/or R² stand for aryl, be achieved in a two-step process, for example by reducing the imine using NaBH₄ (optionally in combination with CoCl₂) and subsequent hydrogenolysis. An example of this two-step process is described by E. J. Corey, Org. Lett. (2000) 2, 1097-1100.

Examples of compounds of formula 6 or salts thereof which can advantageously be prepared with the process of the invention include: allylglycine; propargylglycine; 6-(1,3-dioxolan-2-yl)norvaline; substituted phenylalanines, for example 4-fluoro-, 4-chloro-, 4-bromo, 2-bromo, 3,4-dichloro, 3,4-dihydroxy-, 3-hydroxy-4-methyl- and 4-aryl-substituted phenylalanines; substituted serines; substituted threonines or substituted phenylserines, for example 4-methylthio-, 4-methylsulphonyl- and 4-fluoro-substituted phenylserines; β-mono substituted serines, β,β-disubstituted serines; oligopeptides, for example aspartyl-phenylalanine methyl ester, N-3-fluorobenzyl-glycyl-tert-leucine and leucinyl-tert-leucine N-methylamide; 3-substituted-2,3-diamino carboxylic acids; 4-mono substituted homoserines, 4,4-disubstituted homoserines; substituted aspartic acid (derivatives); substituted glutamic acid (derivatives); substituted γ-cyano-α-aminobutyric acid. γ-Cyano-α-aminobutyric acid is a very interesting compound, since it may be converted to ornithine or proline. Ornithine may subsequently be converted to citrulline or arginine.

Compounds of formula 1 or of formula 6 form excellent substrates for resolution procedures. Resolution procedures are procedures for the separation of enantiomers aimed to obtain an enantiomerically enriched compound.

Various methods known in the art may be employed for the resolution of a compound of formula 1 or of formula 6. For instance, a compound of formula 1 or of formula 6 can be resolved by crystallization induced resolutions, by resolutions via diastereoisomeric salt formation (classical resolutions) or entrainment, for example as described in J. Jacques, A. Collet, S. H. Wilen; ‘Enantiomers, Racemates and Resolutions’, Wiley Interscience, New York (1981). Resolutions can also be achieved, for example, by physical separation methods, for example chiral simulating moving bed as described in ‘Chiral Separation Techniques’, G. Subramanian (Ed.), Wiley, New York (2001), pp 221-251 and 253-285; A. Vande Wouwer, AlChE Journal (2000) 46, 247-256; M. Morbidelli, J Chromatography A (2001) 919, 1-12; and in E. Francotte, Chirality (2002) 14, 313-317. Resolutions can also be achieved, for example, by enzymatic resolutions.

Examples of enzymes which can be used in the enzymatic resolution of compounds of formula 6 wherein X stands for OR⁵, wherein R⁵ is as defined above are stereoselective lipases, for example esterases, for example α-chymotrypsin and subtilisin(alcalase) (for example as described in Can. J. Biochem. (1971) 49, 877 and in ‘Enzym Catalysis in Organic Synthesis’, vol II, K. Drauz, H. Waldmann (Eds.), VCH, Weinheim (2002), pp 398-412).

Examples of enzymes which can be used in the enzymatic resolution of compounds of formula 6 wherein X stands for NR³R⁴, wherein R³ and R⁴ are as defined above are stereoselective amino peptidases or stereoselective amidases. For example, the amino peptidase from Pseudomonas putida ATCC 12633 or the amidase from Ochrobactrum anthropi MIBC 40321 (for example described in ‘Stereoselective Biocatalysis’, R. N. Patel (ed.), Marcel Dekker Inc., New York (2000), pp 23-58), may be used on compounds of formula 6 wherein R³ stands for H and wherein R⁴ stands for H or an alkyl of 14 C-atoms, which alkyl may optionally be substituted or wherein R⁴ stands for an amino acid, an amino acid amide or an N-terminus of a peptide. The peptide might be bound, for example, to a solid phase resin. For example, R⁴ stands for methyl, ethyl, propyl, hydroxyethyl.

Enzymatic resolution may also be performed by stereoselective N-acylation of compounds of formula 6. Or alternatively, a compound of formula 6 may be acylated, after which enzymatic resolution of the formed acylated form of the compound of formula 6 is carried out by a stereoselective acyl hydrolysis reaction. Suitable enzymes in these cases include for example acyl hydrolases also known as acylases, for example penicillin G acylases and Acylase I (for example as described by A. Romeo, J. Org. Chem. (1978) 43, 2576-2581; and in ‘Enzym Catalysis in Organic Synthesis’, vol II, K. Drauz, H. Waldmann (Eds.), VCH, Weinheim (2002), pp 716-760), peptide deformylases (for example as described in EP 1141333) and carbamoylases (for example as described in ‘Enzym Catalysis in Organic Synthesis’, vol II, K. Drauz, H. Waldmann (Eds.), VCH, Weinheim (2002), pp 777-792).

Preferably, the resolution of a compound of formula 1 or of a compound of formula 6 is combined with a racemisation process, for example as described by E. Ebbers et al, Tetrahedron (1997) 53, 9417-9476 in order to obtain a high yield. The racemisation may be performed as a separate process, but is preferably (as is the case in asymmetric transformation or dynamic kinetic resolution) performed in situ. Examples of the combination of the resolution of a compound of formula 6 with a racemisation process are described in D. Kozma, ‘CRC Handbook of Optical Resolution via diastereomeric Salt Formation’, CRC Press, Boca Raton (2002), pp 40-46; R. S. Ward, Tetrahedron Asymm. (1995) 6,1475-1490; and in S. Caddici, K. Jenkins, Chem Soc. Rev. (1996) 28, 447-456.

The invention will now be elucidated by means of the following examples without, however, being limited thereto.

### EXAMPLES

Examples 1-3 show the following reaction:

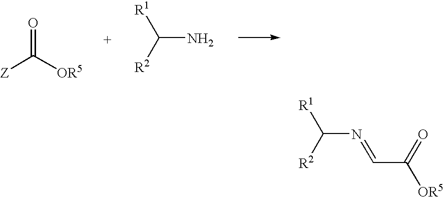

Examples 4-6 show the following reaction:

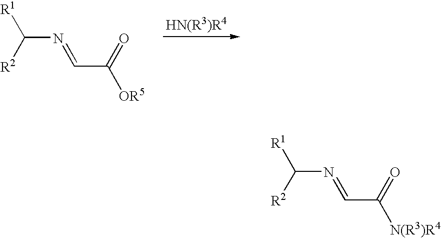

Example 7 and example 10 and example 17 show the following reaction:

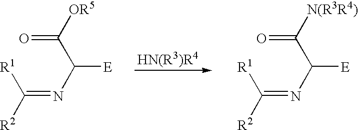

Example 8 shows the direct preparation of 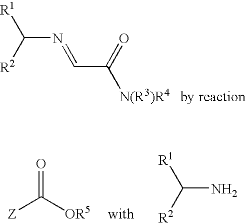
wherein R¹(R²)CH(NH₂) and HN(R³)R⁴ are the same.

Examples 9, 11-15, 16, 17, 20, 18, 21 and 22 show the following reaction:

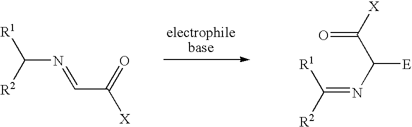

Example 10, example 16, example 17, example 19, example 20 and example 22 show the following reaction:

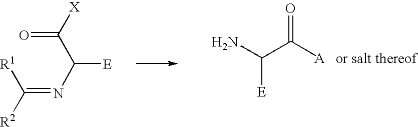

### Example 1

### Reaction of benzhydrylamine with glyoxylic acid methyl ester methyl hemiacetal

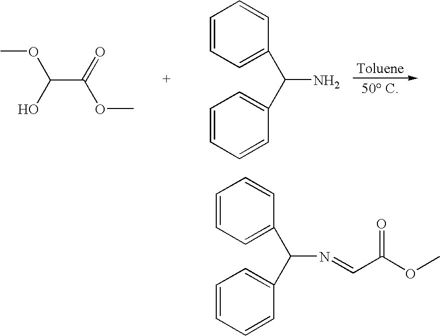

To a solution of glyoxylic acid methyl ester methyl hemiacetal (13.21 g, 110 mmol) in toluene (110 ml, 1M solution) benzhydrylamine (19 ml, 110 mmol, 1 mol eq) was added drop wise. The reaction mixture was heated to 50° C. and stirred under nitrogen. After 1 hour, the reaction mixture was allowed to cool to room temperature and the water layer that had formed was removed. The organic layer was dried over Na₂SO₄, filtered and the solvent removed to yield a colourless oil. Trituration with heptane afforded the product as a white solid in 23.84 g (94.1 mmol, 85.6%). ¹H-NMR (CDCl₃, 300 MHz), δ (ppm): 7.70 (s, 1H, N═CH), 7.20 (m, 10H, 2 C₆H₅), 5.60 (s, 1H, Ph₂CH), 3.79 (s, 3H, OCH₃).

### Example 2

### Reaction of DL-α-methylbenzylamine with glyoxylic acid methyl ester methyl hemiacetal

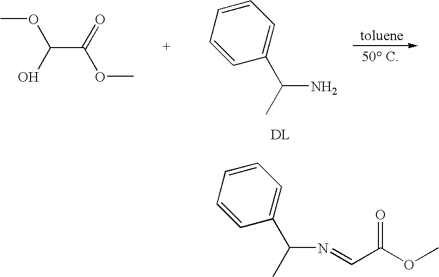

To a solution of glyoxylic acid methyl ester methyl hemiacetal (66 g, 0.55 mol) in toluene (500 ml, 1.1M solution) at 50° C. was added in 10 minutes DL-α-methylbenzylamine (66.7 g, 0.55 mol, 1 mol eq). The reaction mixture was stirred at 50° C. for 1 h under nitrogen. Then it was allowed to cool to room temperature and the water layer that had formed was removed. The organic layer was concentrated under vacuum to yield 100 g (0.52 mol, 95%) of the product as a red oil. ¹H-NMR (CDCl₃, 300 MHz), δ (ppm): 7.75 (s, 1H, N═CH, 7.36-7.25 (m, 5H, C₆H₅), 4.61 (q, 1H, Ph(CH₃)CH), 3.88 (s, 3H, OCH₃), 1.63 (d, 3H, CH₃CHPh).

### Example 3

### Reaction of isopropyl amine with glyoxylic acid methyl ester methyl hemiacetal

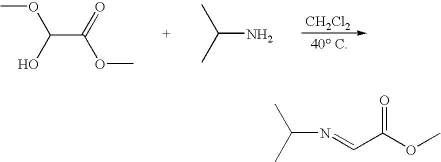

To a solution of glyoxylic acid methyl ester methyl hemiacetal (21.15 g, 176.1 mmol) in CH₂Cl₂ (175 ml, 1M solution) isopropylamine (10.41 g, 15 ml, 176.1 mmol, 1 mol eq) was added. The reaction mixture was then heated to 40° C. and stirred under nitrogen. After 2 h, the reaction mixture was allowed to cool to room temperature and the water layer that had formed was removed. The organic layer was dried over Na₂SO₄, filtered and the solvent removed to yield 19.75 g (152.9 mmol, 87%) of the product as a yellow oil. ¹H-NMR (CDCl₃, 300 MHz), δ (ppm): 7.72 (s, 1H, N═CH, 3.88 (s, 3H, OCH₃), 3.60 (m, 1H, (CH₃)₂CH), 1.27 (d, 6H, (CH₃)₂C).

### Example 4

### Amidation of N-benzydryl-glyoxylic acid imine methyl ester

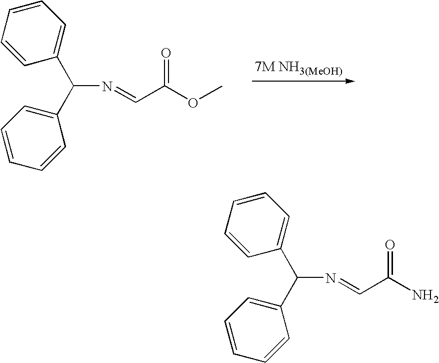

To the N-benzydryl-glyoxylic acid imine methyl ester (20.00 g, 78.9 mmol) a 7M solution of ammonia in methanol (230 ml, 1.61 mol, 20 mol eq.) was added. The resulting suspension was stirred for 10 min. During this time the solid starting material was observed to dissolve rapidly and after 2 min precipitation of a white solid product occurred. After 10 min the suspension was filtered to afford the N-benzydryl-glyoxylic acid imine amide as a white solid in 16.16 g (70.8 mmol, 90%) yield. ¹H-NMR (CDCl₃, 300 MHz), δ (ppm): 7.68 (s, 1H, N═CH), 7.37-7.27 (m, 10H, 2 C₆H₅), 7.08 (br s, 1H, NH, 5.67 (s, 1H, Ph₂CH), 5.41 (br s, 1H, NH).

### Example 5

### Amidation of N-isopropyl-glyoxylic acid imine methyl ester

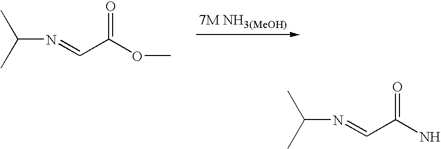

To the N-isopropyl-glyoxylic acid imine methyl ester (2.00 g, 15.5 mmol) a 7M solution of ammonia in methanol (77 ml, 0.539 mol, 35 mol eq.) was added. The solution was stirred for 50 min. The solvent was removed under reduced pressure to afford the product as yellow oil in 1.48 g (13.0 mmol, 84%) yield. ¹H-NMR (CDCl₃, 300 MHz), δ (ppm): 7.56 (s, 1H, N═CH), 6.95 (br s, 1H, NH, 5.42 (br s, 1H, NH), 3.61 (m, 1H, (CH₃)₂CH), 1.22 (d, 6H, 2 CH₃).

### Example 6

### Amidation of N-(1-phenylethyl)-glyoxyl acid imine methyl ester

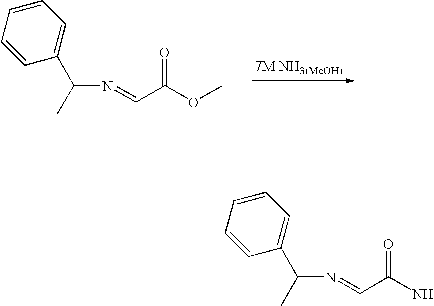

To the N-(1-phenylethyl)-glyoxyl acid imine methyl ester (2.01 g, 10.5 mmol) a 7M solution of ammonia in methanol (39 ml, 0.273 mol, 26 mol eq.) was added. The solution was stirred for 2 h for quantitative conversion (conversion after 30 min was 91%). Then the solvent was removed under reduced pressure to afford the product as brown oil. ¹H-NMR (CDCl₃, 300 MHz), δ (ppm): 7.73 (s, 1H, N═CH), 7.39-7.22 (m, 5H, C₆H₅), 6.99 (br s, 1H, NH), 5.50 (br s, 1H, NH), 4.60 (m, 1H, PhCH), 1.56 (d, 6H, CH₃).

### Example 7

### Amidation of benzophenone imine of glycine methyl ester

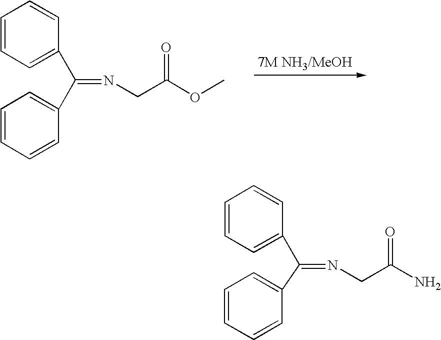

The benzophenone imine of glycine methyl ester (2.02 g, 7.9 mmol) was stirred in 7M NH₃/MeOH solution (39 ml, 0.2M solution) for 20 hours. The reaction mixture was evaporated under reduced pressure and after trituration with pentane the product was obtained as a white solid in 1.57 g (6.6 mmol, 83%) yield. ¹H-NMR (CDCl₃, 300 MHz), δ (ppm): 7.67-7.14 (m, 10H, 2 C₆H₅, 1H, CONH₂), 5.78 (s, 1H, CONH₂), 3.99 (s, 2H, α CH₂).

### Example 8

### Reaction of glyoxylic acid methyl ester, methyl hemiacetal with Excess of isopropylamine

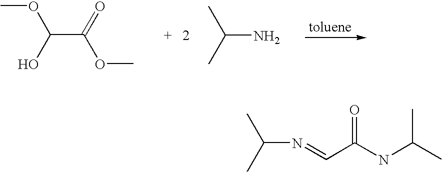

To a stirred solution of glyoxylic acid methyl ester methyl hemiacetal (6.60 g, 55 mmol) in toluene (27.5 ml, 2 M solution), isopropylamine (23.4 ml, 275 mmol, 5 mol eq.) was added drop wise. The temperature of the solution rose to 40° C. After 2 h an additional portion of isopropylamine (18.7 ml, 220 mmol, 4 mol eq.) was added. The reaction mixture was stirred for a further 3 h and then the solvent was removed under reduced pressure to afford an orange coloured oil. Upon standing the oil, the product crystallised as a yellow solid, which was isolated from the liquor and washed with heptane. ¹H-NMR (CDCl₃, 300 MHz), δ (ppm): 7.56 (s, 1H, N═CH), 6.86 (br s, 1 H, CONH), 4.10 (m, 1H, CONHCH), 3.56 (m, 1H, CHN═CH), 1.21 (dd, 12H, (CH₃)₂CH).

### Example 9

### Allylation of N-(1-phenylethyl)-glyoxylic acid imine methyl ester

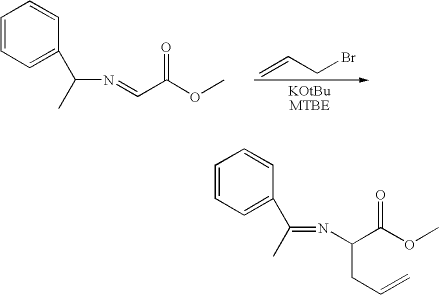

The N-(1-phenylethyl)-glyoxylic acid imine methyl ester (2.00 g, 10.4 mmol) was dissolved in MTBE (30 ml, 0.3M solution) and allylbromide (1.52 g, 1.1 ml, 12.5 mmol, 1.2 mol eq) was added. To this solution KO^{t}Bu (potassium tert-butoxide) (1.29 g, 11.5 mmol, 1.1 mol eq) was added portion-wise over 10 min. An exothermic reaction was noticed as the temperature rose to 40° C. The reaction mixture was stirred under nitrogen for 3.5 h. Then it was washed twice with water. The organic layer was dried over Na₂SO₄, filtered and concentrated under reduced pressure. The product was obtained as a red oil in 1.36 g (5.9 mmol, 56%) yield. ¹H-NMR (CDCl₃, 300 MHz), δ (ppm):7.76-7.72 (m, 2H, orto C₆H₅), 7.32-7.20 (m, 3H, meta and para C₆H₅), 5.82-5.68 (m, 1H, CH₂═CH), 5.10-4.95 (m, 2H, CH₂═CH), 4.35 (m, 1H, α-CH), 3.65 (s, 3H, OCH₃), 2.78-2.52 (2m, 2H, β-CH₂), 2.20 (s, 3H, CH₃CPh).

### Example 10

### Synthesis of DL-allylalycine amide

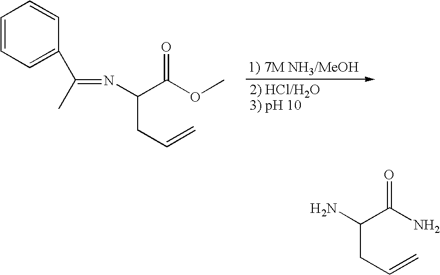

The N-α-methylbenzylidene-DL-allylglycine methyl ester (0.70 g, 3 mmol) was dissolved in 7M NH₃/MeOH solution (15 ml, 0.2M solution) and left stirring for 29 hours. The reaction mixture was then evaporated under reduced pressure, the residue was dissolved in toluene (10 ml) and a 1M aqueous HCl solution (7 ml, 7 mmol, 2.3 mol eq.) was added. The mixture was vigorously stirred for 2 h. The aqueous layer was separated and the pH was adjusted to 10 by addition of 1M NaOH solution. The water layer was extracted with toluene to remove the acetophenone. The aqueous layer was evaporated and the residue suspended in AcOEt. After filtration of the NaCl salt, DL-allylglycine amide was obtained by evaporation of the organic layer under reduced pressure. ¹H-NMR (CDCl₃, 300 MHz), δ (ppm): 7.12 (br s, 1H, CONH) 5.96 (br s, 1H, CONH), 5.69 (m, 1H, CH═CH₂), 5.08 (m, 2H, CH═CH₂), 3.36 (m, 1H, α-CH), 2.52 and 2.24 (2m, 2H, β-CH₂).

### Example 11

### Allylation of N-benzydryl-glyoxyl imine amide

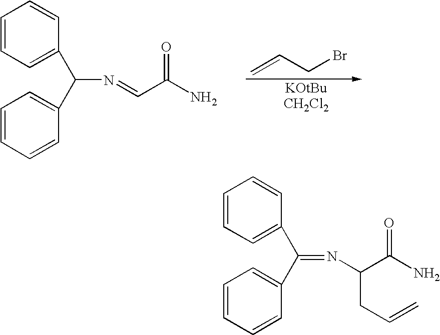

The N-benzydryl-glyoxylic acid imine amide (0.95 g, 4.2 mmol) was suspended in CH₂Cl₂ (20 ml, 0.2M solution) and allylbromide (0.60 g, 0.43 ml, 5.0 mmol, 1.2 mol eq) was added. To this solution KO^{t}Bu (0.52 g, 4.6 mmol, 1.1 mol eq) was added. The reaction mixture was stirred under nitrogen for 3.5 h at room temperature. The reaction mixture was washed twice with water. The aqueous layers were extracted with CH₂Cl₂.

The combined organic layers were dried over Na₂SO₄, filtered and concentrated under reduced pressure. The product was obtained as a yellow oil in 1.02 g (3.7 mmol, 88%) yield. ¹H-NMR (CDCl₃, 300 MHz), δ (ppm): 7.83-7.11 (m, 10H, 2 C₆H₅), 6.83 (br s, 1H, CONH₂), 5.74-5.65 (m, ₁H, vinyl CH═CH₂), 5.54 (br s, 1H, CONH₂), 5.08-5.02 (m, 2H, vinyl CH═CH₂), 4.07 (t, 1H, α-CH), 2.55 (m, 2H, β-CH₂).

### Example 12

### Allylation of N-isopropyl-glyoxylic acid imine methyl ester

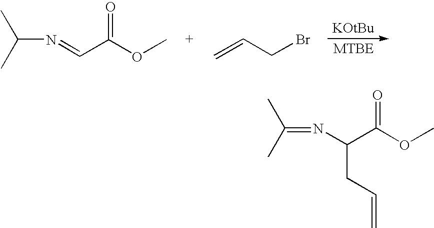

To a solution of N-isopropyl-glyoxylic acid imine methyl ester (1.00 g, 7.74 mmol) in MTBE (30 ml, 0.26 M) was added allylbromide (1.12 g, 0.8 ml, 9.29 mmol, 1.1 mol eq) and KO^{t}Bu (0.95 g, 8.5 mmol, 1.1 mol eq). The mixture was stirred for 15 minutes at room temperature under a N₂ atmosphere. The solvent was removed under reduced pressure. The residue was dissolved in CH₂Cl₂ and the remaining salt (KBr) was filtered on decalite. The organic solution was evaporated under reduced pressure to give the product as a brownish oil in 1.18 g (7.0 mmol, 90%) yield. ¹H-NMR (CDCl₃, 300 MHz), δ (ppm): 5.78 (m, 1H, CH═CH₂), 5.10 (m, 2H, CH═CH₂), 4.17 (m, 1H, α-CH), 3.72 (s, 3H, OCH₃), 2.66 and 2.48 (2m, 2H, β-CH₂), 2.09 and 1.88 (2s, 6H, )CH₃)₂C═N).

### Example 13

### Alkylation of N-isopropyl-glyoxylic acid imine methyl ester with butyliodide

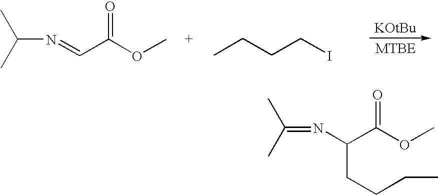

To a solution of N-isopropyl-glyoxylic acid imine methyl ester (1.00 g, 7.7 mmol) in MTBE (30 ml, 0.25M solution) was added butyliodide (4.27 g, 2.64 ml, 23.2 mmol, 3 mol eq), followed by KO^{t}Bu (0.96 g, 8.5 mmol, 1.1 mol eq). The reaction mixture was stirred for 40 min, then the solvent was removed under reduced pressure. The residue was dissolved in CH₂Cl₂ and filtered on decalite to remove the Kl. The organic solution was dried under reduced pressure. The product was obtained as a brownish oil in 1.22 g (6.6 mmol, 85%) yield. ¹H-NMR (CDCl₃, 300 MHz), δ (ppm): 4.05 (m, 1H, α-CH), 3.65 (s, 3H, OCH₃), 2.01 and 1.80 (2s, 6H, (CH₃)₂C═N), 1.90-1.60 (m, 2H, β-CH₂), 1.3-1,1 (m, 4H, γ and δ CH₂), 0.82 (t, 3H, ω-CH₃). cl Example 14

### Alkylation of N-isopronyl-glyoxylic acid imine methyl ester with benzylbromide

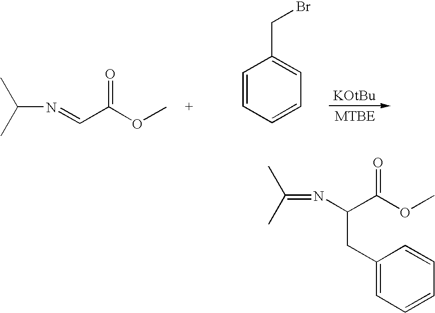

To a stirred solution of N-isopropyl-glyoxylic acid imine methyl ester (1.00 g, 7.7 mmol) in MTBE (30 ml, 0.26 M solution) was added benzylbromide (1.02 ml, 8.5 mmol, 1.1 mol eq.). To the resulting reaction mixture was added KO^{t}Bu (0.87 g, 7.7 mmol, 1 mol eq.) in one portion. The reaction was stirred under N₂ atmosphere for 45 min at room temperature. The solvent was evaporated under reduced pressure, the residue was dissolved in CH₂Cl₂ and the KBr filtered off. The organic solution was evaporated giving the product as a yellow oil in 1.55 g (7.1 mmol, 91%) crude yield. ¹H-NMR (CDCl₃, 300 MHz), δ (ppm): 7.23 (m, 5H, C₆H₅), 4.30 (m, 1H, α-CH), 3.73 (s, 3H, OCH₃), 3.25 and 2.98 (m, 2H, β-CH₂), 2.00 and 1.43 (2s, 6H, (CH₃)₂C═N).

### Example 15

### Cyanoethylation of N-benzydryl-glyoxylic acid imine methyl ester

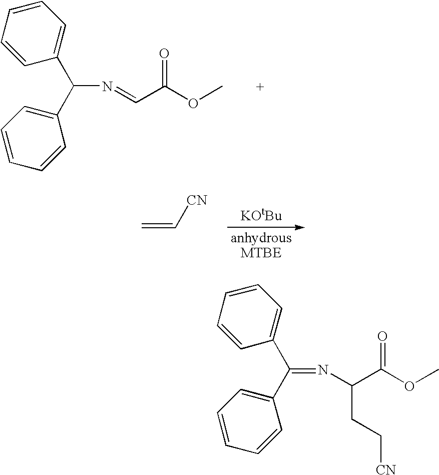

To a stirred solution of N-benzydryl-glyoxylic acid imine methyl ester (1.27 g, 5 mmol) in anhydrous MTBE (20 ml, 0.25 M solution) was added acrylonitrile (0.33 ml, 5 mmol, 1 mol eq.). To the reaction mixture was added KO^{t}Bu (56 mg, 0.5 mmol, 0.1 mol eq.). After 1 hour of stirring an additional portion of acrylonitrile (0.33 ml, 5 mmol, 1 mol eq.) and KO^{t}Bu (0.22 g, 2 mmol, 0.4 mol eq.) was added. The reaction mixture was stirred for 18 hours and then evaporated under reduced pressure to yield the crude product in approximately 90% yield. No work up of the reaction mixture was performed. ¹H-NMR (CDCl₃, 300 MHz), δ (ppm): 7.68-7.19 (m, 10H, 2 C₆H₅), 4.21 (m, 1H, CHCO₂), 3.73 (s, 3H, OCH₃), 2.63-2.23 (m, 4H, CH₂CH₂CN).

### Example 16

### Cyanoethylation of N-(1-phenylethyl)-glyoxylic acid imine methyl ester

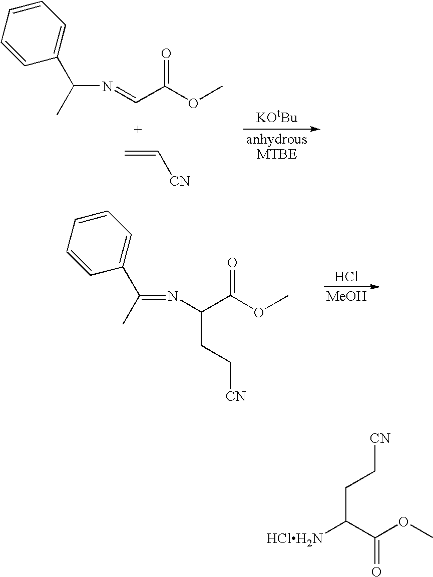

A solution of N-(1-phenylethyl)-glyoxylic acid imine methyl ester (100 g, 0.52 mol) and acrylonitrile (32 g, 0.60 mol. 1.15 mol eq.) in anhydrous 260 ml of MTBE was added in 1 h at 35° C. to a solution of KO^{t}Bu (23.6 g, 0.21 mol) in 400 ml of MTBE. The reaction temperature increased to 46° C. because of the heat of reaction. After 1 h the conversion was estimated to be 98% according to NMR. The reaction mixture was filtrated and evaporated to yield the product as brownish oil. ¹H-NMR (CDCl₃, 300 MHz), δ (ppm): 7.86 (m, 2H, C₆H₅), 7.40 (m, 3H, C₆H₅), 4.54 (t, 1H, CHCO₂CH₃), 3.75 (s, 3H, OCH₃), 2.54 (t, 2H, CH₂CH₂CN), 2.40-2.34 (t+s, 5H, CH₂CH₂CN+CH₃).

The crude product was dissolved in methanol and hydrolysed at 20° C. for 1 h using 1 equivalent of conc. HCl solution. After evaporation of the methanol, the acetophenone and cyanoethylglycine methyl ester HCl-salt were separated in toluene/water. Evaporation of the aqueous layer gave the cyanoethylglycine methyl ester HCl-salt in quantitative conversion. ¹H-NMR (DMSO-d₆, 300 MHz), δ (ppm): 9.0 (br s, 3H, NH₃⁺), 4.08 (m, 1H, CHCO₂CH₃), 3.78 (s, 3H, OCH₃), 2.90 (m, 2H, CH₂CH₂CN), 2.19 (m, 2H, CH₂CH₂CN).

### Example 17

### Synthesis of DL-cyanoethylglycine amide HCl Salt

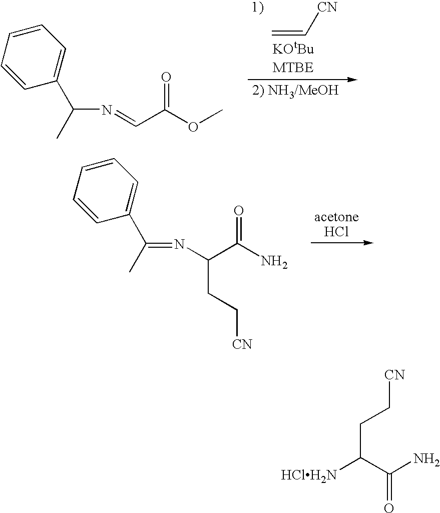

To a stirred solution of N-(1-phenylethyl)-glyoxylic acid imine methyl ester (10.24 g, 53.5 mmol) in anhydrous MTBE (150 ml, 0.36 M solution) was added acrylonitrile (7.0 ml, 107.1 mmol, 2 mol eq.). To the resulting reaction mixture was added KO^{t}Bu (3.00 g, 26.7 mmol, 0.5 mol eq.) portion-wise over 10 min. After stirring for 1 hour, an additional portion of acrylonitrile (3.5 ml, 53.5 mmol, 1 mol eq.) was added. After 22 h the reaction mixture was filtered and the solvent removed under reduced pressure to give the crude N-α-methylbenzylidene-DL-cyanoethylglycine methyl ester as a brownish oil in 8.36 g (34.2 mmol, 64%) yield. ¹H-NMR (CDCl₃, 300 MHz), δ (ppm): 7.85 (d, 2H, orto C₆H₅), 7.43-7.15 (m, 3H, meta and para C₆H₆), 4.52 (m, 1H, CHCO₂CH₃), 3.75 (s, 3H, OCH₃), 2.61-2.28 (m, 4H, CH₂CH₂), 2.35 (s, 3H, CH₃).

To the oil was added 200 ml of 7 M NH₃ solution in MeOH. The resulting solution was stirred for 24 h after which the solvent was evaporated under reduced pressure. The crude oil obtained was dissolved in acetone (150 ml, 0.36 M solution based on 100% conversion in previous steps) and to that solution was added a concentrated aqueous solution of HCl (37 wt %, 6.6 ml, 80.2 mmol). The mixture was stirred for 40 min. During this time a white solid formed. The suspension was filtered to afford the DL-cyanoethyl glycine amide hydrochloride salt as a white solid. ¹H-NMR (d₆-DMSO, 300 MHz), δ (ppm): 8.45 (br s, 3H, NH₃⁺), 8.11 (br s, 1H, NH), 7.66 (br s, 1H, NH), 3.81 (br m, 1H, CHCONH₂), 2.68 (t, 2H, CH₂CN), 2.10 (m, 2H, CHCH₂).

### Example 18

### Propargylation of N-benzydryl-glyoxylic acid imine amide

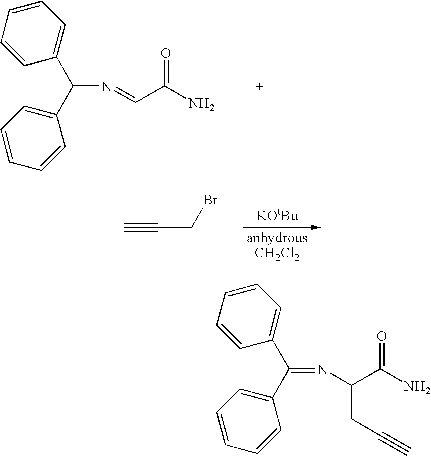

To a stirred suspension of the N-benzydryl-glyoxylic acid imine amide (10.00 g, 43.8 mmol) in anhydrous CH₂Cl₂ (200 ml, 0.22 M solution) was added an 80 wt % solution of propargylbromide in toluene (4.5 ml, 52.5 mmol, 1.2 mol eq.). To the resulting reaction mixture was added KO^{t}Bu ( 5.40 g, 48.2 mmol, 1.1 mol eq.) portion wise over 15 min. The reaction temperature rose to 37° C. After stirring for 1 hour at room temperature an additional portion of 80 wt % propargylbromide solution (3.8 ml, 43.8 mmol 1 mol eq.) and KO^{t}Bu (2.95 g, 26.3 mmol, 0.6 mol eq.) was added. Again the reaction temperature rose (to 30° C.). The reaction mixture was stirred for an additional hour and then was washed with water (3×100 ml). The organic layer was dried (Na₂SO₄), filtered and the solvent was removed under reduced pressure to afford N-(diphenylmethylene)-DL-propargylglycine amide as a brownish oil in 10.26 g (37.1 mmol, 85%) yield. ¹H-NMR (CDC₃, 300 MHz), δ (ppm): 7.69 (d, 2H, C₆H₅), 7.51-7.16 (m, 8H, C₆H₅), 6.76 (br s, 1H, NH), 5.56 (br s, 1H, NH), 4.15 (dd, 1H, CHCONH₂), 2.80-2.61 (m, 2H, CH₂), 1.99 (t, 1H, CCH).

### Example 19

### Acidic hydrolysis of the benzophenone imine to propargylglycine amide HCl Salt

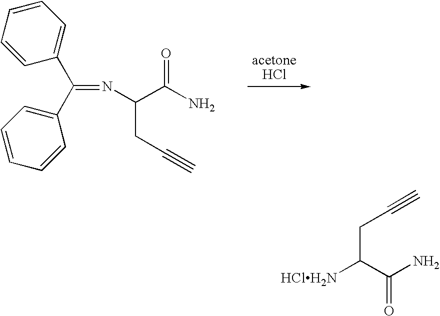

To a stirred solution of N-(diphenylmethylene)-DL-propargylglycine amide (10.26 g, 37.1 mmol) in acetone (100 ml, 0.37 M solution) was added concentrated aqueous HCl (37 wt %, 5.4 ml, 65.7 mmol, 1.7 mol eq.). The reaction mixture became dark coloured in 2 min and after 15 min a white solid precipitate formed. The reaction was stirred for a further 30 min and then the solid was isolated by filtration. This afforded DL-propargylglycine amide HCl salt as a white solid in 3.30 g (22.2 mmol, 60%) yield. ¹H-NMR (d₆-DMSO, 300 MHz), δ (ppm): 8.37 (br s, 3H, NH₃⁺), 7.98 (br s, 1H, NH), 7.63 (br s, 1H, NH), 3.86 (br m, 1H, CHCONH₂), 3.12 (s, 1H, CCH), 2.87-2.70 (m, 2H, CH₂).

### Example 20

### Synthesis of DL-allylglycine amide HCl Salt under PTC Conditions

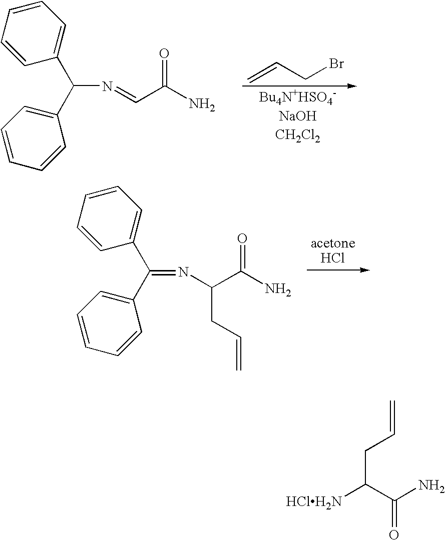

To a stirred suspension of N-benzydryl-glyoxylic acid imine amide (2.00 g, 8.7 mmol) in CH₂Cl₂ (35 ml, 0.25 M solution) was added the phase transfer catalyst Bu₄N⁺HSO₄⁻ (0.30 g, 0.9 mmol, 0.1 mol eq.) and 8M NaOH solution (2.2 ml, 17.5 mmol, 2 mol eq.). To this vigorously stirred mixture allylbromide (0.8 ml, 9.6 mmol, 1.1 mol eq.) was added. After stirring for 17 h at room temperature, 40 ml of water were added and the two layers were separated. The aqueous layer was extracted with CH₂Cl₂. After washing the combined organic layers with water, the solvent was removed under reduced pressure. The residue was dissolved in acetone (20 ml) and concentrated aqueous HCl (37%, 1.0 ml, 13.1 mmol) was added. The mixture was stirred for 45 min and then the solid DL-allylglycine amide HCl salt was isolated by filtration. ¹H-NMR (d₆-DMSO, 300 MHz), δ (ppm): 8.24 (br s, 3H, NH₃⁺), 7.93 (br s, 1H, NH), 7.55 (br s, 1H, NH), 5.76 (m, 1H, γ-CH), 5.17 (m, 2H, δ-CH₂), 3.80 (m, 1H, CHCONH₂), 3.50 (m, 2H, β-CH₂).

### Example 21

### Alkylation of N-(1-phenylethyl)-glyoxylimine methyl ester with crotonitrile

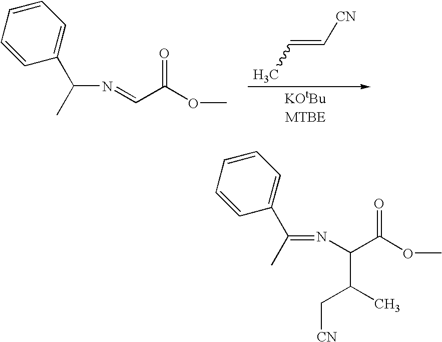

The N-(1-phenylethyl)-glyoxylimine methyl ester (1.00 g, 5.2 mmol) was dissolved in MTBE (20 ml, 0.26M solution) and crotonitrile (0.35 g, 0.42 ml, 5.2 mmol, 1 mol eq) was added. To this solution KO^{t}Bu (0.29 g, 2.6 mmol, 0.5 mol eq) was added at once. An exothermic reaction was noticed as the temperature rose to 33° C. The reaction mixture was stirred under nitrogen for 2.5 h. Then the reaction mixture was filtered and the solvent removed under reduced pressure to give the crude product as a yellow oil in 0.91 g (3.5 mmol, 68%) yield as a 60:40 diastereomeric mixture.

¹H-NMR (CDCl₃, 300 MHz), δ (ppm): 7.78 (m, 2H, ortho C₆H₅), 7.34 (m, 3H, meta and para C₆H₅), 4.36 and 4.18 (2xd, 1H, CHCO₂CH₃), 3.67 (s, 3H, OCH₃), 2.70-2.35 (m, 3H, CHCH₂CN), 2.26 and 2.21 (2xs, 3H, CH₃CPh), 1.16 and 1.12 (2xd, 3H, CHCHCH₃).

### Example 22

### Synthesis of DL-diphenylalanine amide HCl Salt under Phase Transfer Catalyst Conditions

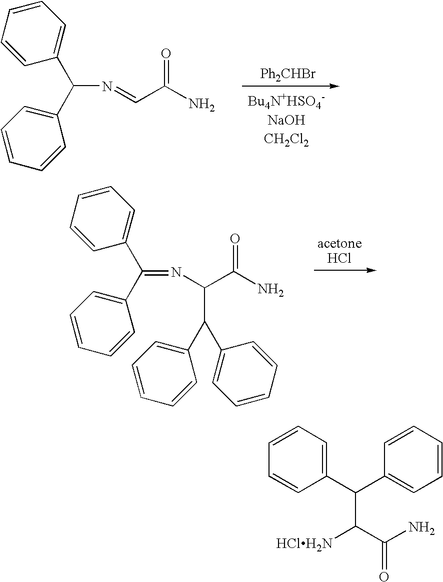

To a suspension of N-benzydryl-glyoxylic acid imine amide (25.0 g, 105 mmol) in CH₂Cl₂ (250 ml), is added a 32% NaOH solution (262 g, 2.1 mol, 20 eq.) and Bu₄N⁺HSO₄⁻ (3.56 g, 10.5 mmol, 0.1 eq.) at room temperature. Then diphenylmethylbromide (28.5 g, 115 mmol, 1.1 eq.) is added in one portion. The mixture is vigorously stirred at room temperature until complete conversion (3.5 h). Then the reaction mixture is diluted with water (250 ml) and with CH₂Cl₂ (750 ml). The phases are separated and the organic layer is washed 3 times with water (150 ml each) and with an aqueous saturated solution of ammonium chloride (150 ml). The organic layer is concentrated in vacuo at 40° C. to dryness. The remaining compound (46.9 g) is suspended in acetone (105 ml), then concentrated aqueous HCl (37%, 20.7 g, 210 mmol, 2 eq.) is added. The reaction mixture is stirred at room temperature until complete conversion (2-3 h), then the precipitate is filtered off. The product is dried at 40° C. under vacuo to constant weight to yield 23.1 g (79.5%) of a white powder.

1H-NMR (d₆-DMSO, 300 MHz), δ (ppm): 8.36 (s, 3H), 8.11 (s, 1H), 7.19-7.34 (m, 11H), 4.90 (m, 1H), 4.32 (d, 1H).

1. Process for the preparation of an (x-amino-carbonyl compound of formula 1, 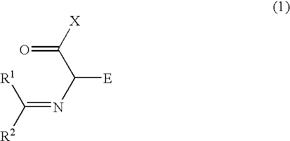
wherein R¹ and R² each independently stand for optionally substituted (cyclo)alkyl, optionally substituted (cyclo)alkenyl, optionally substituted (hetero)aryl, CN or C(O)R⁶, —wherein R⁶ stands for OR¹²—, —wherein R¹² stands for an optionally substituted (cyclo)alkyl, an optionally substituted aryl- or wherein R⁶ stands for NR¹³R¹⁴, —wherein R¹³ and R¹⁴ are each independently chosen from the group of H, optionally substituted (cyclo)alkyl and optionally substituted (hetero)aryl and wherein R¹³ and R¹⁴ may form a ring together with the N-atom to which they are connected- and wherein R¹ and/or R² may be part of a ring system formed by a connection between R¹ and R², between R¹ and E, between R² and E, between R¹ and X or between R² and X, wherein X and E are as defined below,
wherein E stands for H, an optionally substituted (cyclo)alkyl, a halogen, a tri-substituted silyl group, an optionally substituted (cyclo)alkenyl, an optionally substituted (hetero)aryl or wherein E stands for C(O)R⁴⁰, —wherein R⁴⁰ stands for H, an optionally substituted (cyclo)alkyl, an optionally substituted (hetero)aryl or for OR⁴¹, —wherein R⁴¹ stands for an optionally substituted (cyclo)alkyl or an optionally substituted (hetero)aryl or wherein R⁴⁰ stands for NHR⁴², —wherein R⁴² stands for H, an optionally substituted (cyclo)alkyl or for an optionally substituted aryl-,
and wherein X stands for OR⁵, —wherein R⁵ stands for an optionally substituted (cyclo)alkyl, an optionally substituted aryl- or wherein X stands for NR³R⁴, —wherein R³ and R⁴ each independently stand for H, an optionally substituted (cyclo)alkyl or an optionally substituted (hetero)aryl and wherein R³ and R⁴ may form a ring together with the N-atom to which they are bound-,
and wherein X together with E may form part of a lactone or lactam ring system together with the C-atoms to which they are bound, characterized in that an imine of formula 2, 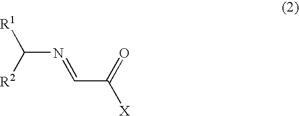
wherein R¹, R² and X are as defined above, is reacted with a suitable electrophile in the presence of a base to form the corresponding a-amino carbonyl compound of formula 1.

2. Process according to claim 1, characterized in that R¹ and R² each independently stand for optionally substituted (cyclo)alkyl, optionally substituted (cyclo)alkenyl, optionally substituted (hetero)aryl, wherein R¹ and/or R² may be part of a ring system formed by a connection between R¹ and R², between R¹ and E, between R² and E, between R¹ and X or between R² and X, wherein X and E are as defined above.

3. Process according to claim 2, characterized in that R¹ and R² each independently stand for an optionally substituted (cyclo)alkyl or an optionally substituted (hetero)aryl, wherein R¹ and R² may be part of a ring system formed by a connection between R¹ and R².

4. Process according to claim 1, characterized in that X stands for OR⁵, wherein R⁵ stands for an optionally substituted (cyclo)alkyl or X stands for NR³R⁴, wherein R³ and R⁴ each independently stand for H, optionally substituted (cyclo)alkyl or optionally substituted aryl, wherein R³ and R⁴ may form a ring together with the N-atom to which they are bound, and wherein X together with E may form part of a lactone or lactam ring system together with the C-atoms to which they are bound.

5. Process according to claim 1, characterized in that E stands for H or an optionally substituted (cyclo)alkyl, wherein E together with X may form part of a lactone or lactam ring system together with the C-atoms to which they are bound

6. Process according to claim 1, characterized in that the process is performed in an anhydrous organic solvent.

7. Process according to claim 1, characterized in that the process is performed in a two-phase system in the presence of a phase transfer catalyst.

8. Process according to claim 7, characterized in that the phase transfer catalyst is chiral and enantiomerically enriched.

9. Process according to claim 1, characterized in that the compound of formula 2 has a chiral group.

10. Process according to claim 9, characterized in that X stands for a chiral group.

11. Process according to claim 9, characterized in that the compound of formula 2 is also enantiomerically enriched.

12. Process according to claim 1, characterized in that a compound of formula 2, wherein X stands for OR⁵, wherein R⁵ is as defined above, is prepared by reacting a glyoxylic acid ester (derivative) of formula 3, 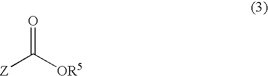
wherein Z is CHO or a masked aldehyde group, with an amine of formula 4, 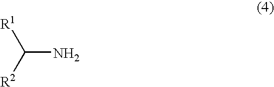
wherein R¹ and R² are as defined above.

13. Process according to claim 1, characterized in that a compound of formula 2, wherein X stands for NR³R⁴, wherein R³ and R⁴ are as defined above is prepared by further reacting the imine of a glyoxylic acid compound of formula 2, wherein X stands for OR⁵, wherein R⁵ is as defined above with an amine of formula 5, 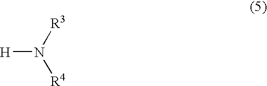
wherein R³ and R⁴ are as defined above.

14. Process according to claim 13, characterized in that NR³R⁴ stands for an amino acid ester, an amino acid amide, an amino nitrile or for an N-terminus of a peptide.

15. Process according to claim 13, characterized in that the compound of formula 4 and the compound of formula 5 are the same.

16. Process according to claim 1, characterized in that an α-amino carbonyl compound of formula 1, wherein X stands for OR⁵, wherein R⁵ is as defined above, is further reacted with an amine of formula 5, 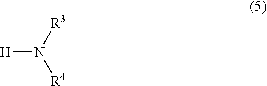
wherein R³ and R⁴ are as defined above to form the corresponding α-amino carbonyl compound of formula 1 wherein X stands for NR³R⁴, wherein R³ and R⁴ are as defined above.

17. Process according to claim 1, characterized in that an α-amino carbonyl compound of formula 1 is further converted to form the corresponding compound of formula 6 or a salt thereof, 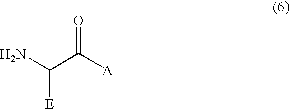
wherein A stands for OH or X and wherein X and E are as defined above, in a manner known per se.

18. Process according to claim 1, characterized in that a compound of formula 1 or a compound of formula 6 is subjected to a crystallization induced resolution, to a resolution via diastereomeric salt formation or entrainment or to a physical separation method.

19. Process according to claim 1 characterized in that a compound of formula 6 or the acylated form of the compound of formula 6 is subjected to enzymatic resolution.

20. Process according to claim 19, characterized in that the compound of formula 6 is subjected to enzymatic resolution by stereoselective N-acylation of the compound of formula 6 or in that the compound of formula 6 is first acylated after which the formed acylated form of the compound of formula 6 is subjected to enzymatic resolution.

21. Process according to claim 19, characterized in that the compound of formula 6, wherein R³ stands for H and wherein R⁴ stands for H or an optionally substituted alkyl of 1-4 C-atoms, is subjected to enzymatic resolution by using a stereoselective amino peptidase or a stereoselective amidase.

22. Process according to claim 19, characterized in that the resolution is combined with a separate or in situ racemisation process.

23. Process according to claim 22, characterized in that the resolution combined with a racemisation process is asymmetric transformation or dynamic kinetic resolution.

24. Process according to claim 1, wherein the compound of formula 6 is γ-cyano-α-aminobutyric acid and wherein γ-cyano-α-aminobutyric acid is subsequently converted to form ornithine, citrulline, arginine or proline.
